SEQUENCE LISTING (8 sequences)

SEQ ID NO: 1 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 2 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 3 - protein, 73 aa, Homo sapiens
RQDPESLPFRQPVDPQLLGIPDYFDIVKNPMDLSTIKRKLDTGQYQEPWQYVDDVWLMFNNAWLYNRKTSRVY

SEQ ID NO: 4 - protein, 73 aa, Homo sapiens
RQDPESLPFRQPVDPQLLGIPDYFDIVKSPMDLSTIKRKLDTGQYQEPWQYVDDIWLMFNNAWLYNRKTSRVY

SEQ ID NO: 5 - protein, 116 aa, Homo sapiens
KKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLG

SEQ ID NO: 6 - protein, 122 aa, Homo sapiens
APGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLG

SEQ ID NO: 7 - protein, 380 aa, Homo sapiens
ENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 8 - protein, 381 aa, Homo sapiens
ENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQD

(19) United States
(12) Patent Application Publication
(10) Pub. No.: US 2017/0196878 A1
(43) Pub. Date: Jul. 13, 2017

(54) USE OF CBP/EP300 AND BET INHIBITORS FOR TREATMENT OF CANCER

(71) Applicants: GENENTECH, INC., South San Francisco, CA (US); CONSTELLATION PHARMACEUTICALS, INC., Cambridge, MA (US)

(72) Inventors: Richard C. Centore, Cambridge, MA (US); Andrew R. Conery, Cambridge, MA (US); Karen Gascoigne, South San Francisco, CA (US); Robert J. Sims, III, Cambridge, MA (US)

(73) Assignees: GENENTECH, INC., South San Francisco, CA (US); CONSTELLATION PHARMACEUTICALS, INC., Cambridge, MA (US)

(21) Appl. No.: 15/463,477

(22) Filed: Mar. 20, 2017

Related U.S. Application Data
(63) Continuation of application No. PCT/US2015/050877, filed on Sep. 18, 2015.
(60) Provisional application No. 62/052,987, filed on Sep. 19, 2014.

(51) Int. Cl. A61K 31/551; A61K 31/5377

(52) U.S. Cl. CPC A61K 31/551; A61K 31/5377

(57) ABSTRACT

The present invention relates to the use of CBP/EP300 inhibitors and BET inhibitors for the treatment of cancer. In some embodiments, the use is to treat cancer that is resistant to BET inhibitors.

### CROSS REFERENCE TO RELATED APPLICATION(S)

This patent application claims the benefit of priority of U.S. application Ser. No. 62/052,987, filed Sep. 19, 2014, which application is herein incorporated by reference.

### TECHNICAL FIELD

The present invention relates to use of CBP/EP300 inhibitors and BET inhibitors for the treatment of cancer.

### BACKGROUND

Chromatin is a complex combination of DNA and protein that makes up chromosomes. It is found inside the nuclei of eukaryotic cells and is divided between heterochromatin (condensed) and euchromatin (extended) forms. The major components of chromatin are DNA and proteins. Histones are the chief protein components of chromatin, acting as spools around which DNA winds. The functions of chromatin are to package DNA into a smaller volume to fit in the cell, to strengthen the DNA to allow mitosis and meiosis, and to serve as a mechanism to control expression and DNA replication. The chromatin structure is controlled by a series of post-translational modifications to histone proteins, notably histones H3 and H4, and most commonly within the “histone tails” which extend beyond the core nucleosome structure. Histone tails tend to be free for protein-protein interaction and are also the portion of the histone most prone to post-translational modification. These modifications include acetylation, methylation, phosphorylation, ubiquitinylation, SUMOylation. These epigenetic marks are written and erased by specific enzymes that place the tags on specific residues within the histone tail, thereby forming an epigenetic code, which is then interpreted by the cell to allow gene specific regulation of chromatin structure and thereby transcription.

Of all classes of proteins, histones are amongst the most susceptible to post-translational modification. Histone modifications are dynamic, as they can be added or removed in response to specific stimuli, and these modifications direct both structural changes to chromatin and alterations in gene transcription. Distinct classes of enzymes, namely histone acetyltransferases (HATS) and histone deacetylases (HDACs), acetylate or de-acetylate specific histone lysine residues (Struhl K., Genes Dev., 1989, 12, 5, 599-606).

Covalent modification of histones is a fundamental mechanism of control of gene expression, and one of the major epigenetic mechanisms at play in eukaryotic cells (Kouzarides, Cell, 128, 693-705 (2007)). Because distinct transcriptional states define fundamental cellular processes, such as cell type specification, lineage commitment, cell activation and cell death, their aberrant regulation is at the core of a range of diseases (Medzhitov et al., Nat. Rev. Immunol., 9, 692-703 (2009); Portela et al., Nat. Biotech., 28, 1057-1068 (2010)). A fundamental component of the epigenetic control of gene expression is the interpretation of histone modifications by proteins that harbor specialized motifs that bind to such modifications. Among them, bromodomains have evolved to bind to acetylated histones and by so doing they represent fundamental links between chromatin structure and gene transcription (Fillipakoppoulos et al., Cell, 149, 214-231 (2012)).

Bromodomains, which are approximately 110 amino acids long, are found in a large number of chromatin-associated proteins and have been identified in approximately 70 human proteins, often adjacent to other protein motifs (Jeanmougin F., et al., Trends Biochem. Sci., 1997, 22, 5, 151-153; and Tamkun J. W., et al., Cell, 1992, 7, 3, 561-572). Interactions between bromodomains and modified histones may be an important mechanism underlying chromatin structural changes and gene regulation. Bromodomain-containing proteins have been implicated in disease processes including cancer, inflammation and viral replication. See, e.g., Prinjha et al., Trends Pharm. Sci., 33(3):146-153 (2012); Muller et al., Expert Rev., 13(29):1-20 (September 2011); and Wyce et al., Oncotarget, 4(12):2419-2429 (2013).

Cell-type specificity and proper tissue functionality requires the tight control of distinct transcriptional programs that are intimately influenced by their environment. Alterations to this transcriptional homeostasis are directly associated with numerous disease states, most notably cancer, immuno-inflammation, neurological disorders, and metabolic diseases. Bromodomains reside within key chromatin modifying complexes that serve to control distinctive disease-associated transcriptional pathways. This is highlighted by the observation that mutations in bromodomain-containing proteins are linked to cancer, as well as immune and neurologic dysfunction. Hence, the selective inhibition of bromodomains across the family creates varied opportunities as novel therapeutic agents in human dysfunction.

There is a need for treatments for cancer and other bromodomain related diseases.

### SUMMARY

One aspect of the present invention provides a method for treating or delaying progression of cancer in an individual comprising administering an effective amount of a CBP/EP300 inhibitor and a BET inhibitor to the individual.

In certain embodiments, the CBP/EP300 inhibitor and the BET inhibitor are concomitantly administered.

In certain embodiments, the CBP/EP300 inhibitor and the BET inhibitor are coformulated.

In certain embodiments, wherein the CBP/EP300 inhibitor is administered separately from the BET inhibitor.

In certain embodiments, wherein the CBP/EP300 inhibitor is administered sequentially with the BET inhibitor.

In certain embodiments, wherein the CBP/EP300 inhibitor is administered simultaneously with the BET inhibitor.

In certain embodiments, wherein the individual is administered the BET inhibitor and subsequently administered the CBP/EP300 inhibitor.

In certain embodiments, the individual is administered the CBP/EP300 inhibitor and subsequently administered the BET inhibitor.

In certain embodiments, administration of the CBP/EP300 inhibitor and the BET inhibitor slows growth of cancer cells to a greater extent than administration of either inhibitor alone.

Another aspect of the present invention provides a method of treating or delaying progression of cancer, wherein the cancer is resistant to a BET inhibitor, in an individual comprising administering an effective amount of a CBP/EP300 inhibitor to the individual.

In certain embodiments, the cancer is selected from acoustic neuroma, acute leukemia, acute lymphocytic leukemia, acute myelocytic leukemia, acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer (NSCLC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In certain embodiments, the cancer is a B-cell proliferative cancer.

In certain embodiments, the cancer is leukemia or lymphoma.

In certain embodiments, the cancer is leukemia.

In certain embodiments, the cancer is breast cancer.

In certain embodiments, the cancer is myeloma.

In certain embodiments, the individual is human.

In certain embodiments, the CBP/EP300 inhibitor is a HAT domain inhibitor.

In certain embodiments, the CBP/EP300 inhibitor is a bromodomain inhibitor.

In certain embodiments, the CBP/EP300 inhibitor inhibits CBP.

In certain embodiments, the EP300 inhibitor inhibits EP300.

Another aspect of the present invention provides a CBP/EP300 inhibitor and BET inhibitor combination for use in medical treatment or diagnosis including therapy and/or treating cancer.

Another aspect of the present invention provides a CBP/EP300 inhibitor for use in medical treatment or diagnosis including therapy and/or treating cancer, wherein the cancer is resistant to a BET inhibitor.

In certain embodiments, the cancer is selected from acoustic neuroma, acute leukemia, acute lymphocytic leukemia, acute myelocytic leukemia, acute t-cell leukemia, B-cell proliferative cancer, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer (NSCLC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. In certain embodiments, the cancer is a B-cell proliferative cancer. In certain embodiments, the cancer is leukemia or lymphoma. In certain embodiments, the cancer is leukemia. In certain embodiments, the cancer is myeloma. In certain embodiments, the cancer is breast cancer.

In certain embodiments, the CBP/EP300 inhibitor inhibits CBP. In certain embodiments, the CBP/EP300 inhibitor inhibits EP300. In certain embodiments, the CBP/EP300 inhibitor inhibits the bromodomain. In certain embodiments, the CBP/EP300 inhibitor inhibits the histone acetyltransferase domain (HAT domain). In certain embodiments, the CBP/EP300 inhibitor binds the HAT domain of CBP and/or EP300. In certain embodiments, the CBP/EP300 inhibitor binds the bromodomain of CBP and/or EP300.

In certain embodiments of any of the methods, the individual is a human, e.g., a female or male.

One aspect of the present invention a CBP/EP300 inhibitor for use in medical treatment or diagnosis including therapy and/or treating cancer.

### BRIEF DESCRIPTION OF THE FIGURES

FIG. 1. Synergistic effect of CBP/EP300 inhibitor and BET inhibitor in leukemia cell lines.

FIG. 2. Synergistic effect of CBP/EP300 inhibitor and BET inhibitor in breast cancer cell lines.

FIG. 3. Generation of BET inhibitor resistant cells.

FIG. 4. Dysfunctional apoptosis in BET inhibitor resistant cells.

FIG. 5. BET inhibitor resistant cells maintain MYC expression.

FIG. 6. CBP/EP300 bromodomains are required for MYC expression in BET inhibitor resistant cells.

FIG. 7. CBP/EP300 bromodomain inhibitors suppress MYC and inhibit growth.

FIG. 8. CBP/EP300 and BET bromodomain inhibition have distinct transcriptional effects. A, LP-1 cells were treated with SGC-CBP30 (2.5 μM) or CPI203 (0.25 μM) for 6 hours, and mRNA expression was measured using RNA sequencing. Expression values for replicate compound treated samples were normalized to paired DMSO controls to obtain log2 fold change values. B, Example enrichment plots for GSEA of SGC-CBP30 treated LP-1 cells. C, The instances of gene sets in the c2 database (MSigDB) related to MYC or multiple myeloma+IRF4 in ranked lists (by NES) of significantly enriched gene sets for SGC-CBP30 or CPI203 treatment are shown. D, IRF4 target genes differentially expressed (minimum 1.5 fold, p<0.05) with SGC-CBP30, but not CPI203. E, Dose-dependent inhibition of IRF4 mRNA expression with SGC-CBP30 in LP-1. Cells were treated with compound for 6 h, and mRNA expression was assessed with q-RTPCR and normalized to GAPDH. Values represent the mean of n=3, ±SEM. F, BET inhibition does not directly regulate IRF4 expression. LP-1 cells were treated with a titration of CPI203 for 6 h, and IRF4 expression was determined by q-RTPCR and normalized to GAPDH. Values represent the mean of n=2, ±SEM.

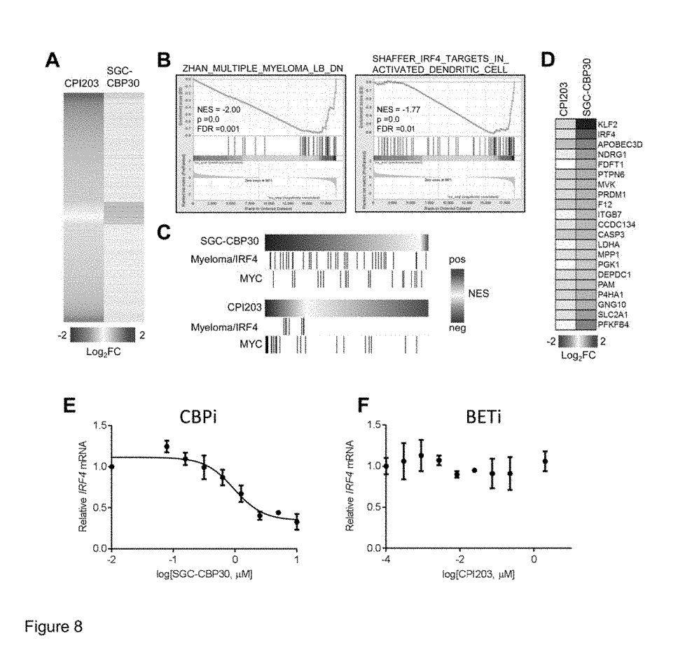

FIG. 9. CBP/EP300 bromodomain inhibition enhances the phenotypic effects of low dose BET bromodomain inhibition. The indicated cell lines were treated with a titration of CPI-267203 in the presence of DMSO or CPI-529552 (3.33 μM or 10 μM). Plots of % Growth (top) were obtained by counting viable cells and normalizing to cell counts in the DMSO treated condition at each concentration of CPI-529552. Plots of % subG1 were obtained by counting cells with less than G1 DNA content.

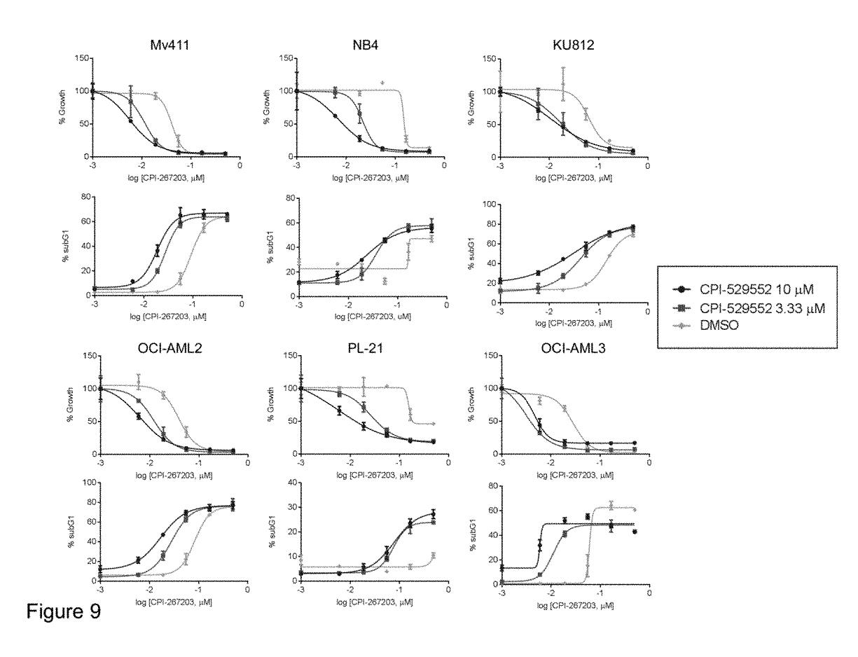

FIG. 10. Combination of CBP/EP300 and BET bromodomain inhibitors enhances apoptosis in a multiple myeloma cell line. AMO-1 cells were treated with DMSO, low dose CBPi (1.2 μM CPI778), high dose CBPi (6 μM CPI778), low dose BETi (0.05 μM CPI203), high dose BETi (0.25 μM CPI203), or low dose CBPi+low dose BETi. Cells were fixed and analyzed for viable cell number (top) or % subG1 (bottom) at the indicated time points.

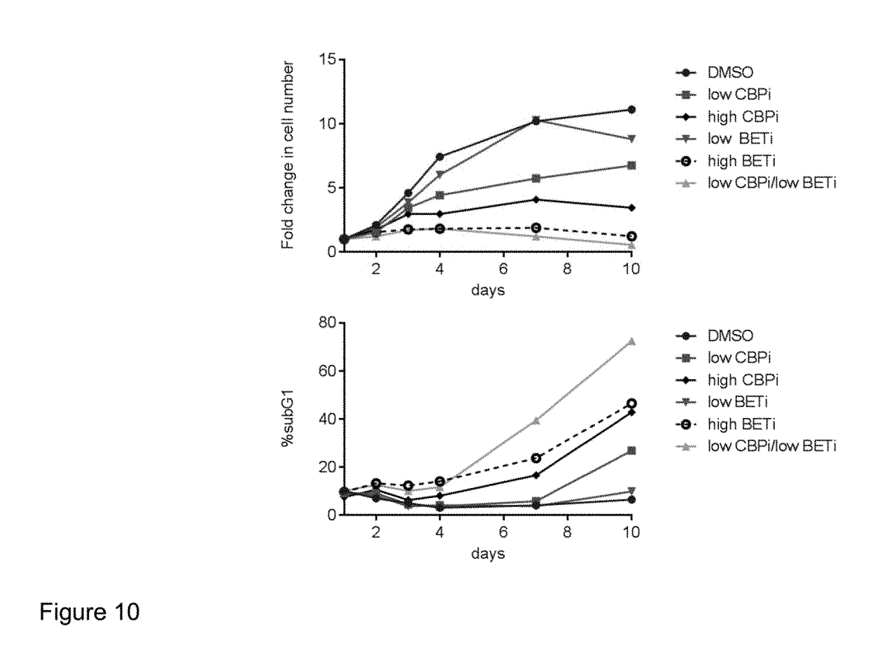

FIG. 11. Combination of CBP/EP300 and BET bromodomain inhibitors enhances the suppression of soft agar colony formation by breast cancer cell lines. The indicated cell lines were plated in soft agar and treated with DMSO, high dose BETi (0.25 μM CPI203), high dose CBPi (0.175 μM CPI821), low dose BETi (0.04 μM CPI203), low dose CBPi (0.09 μM CPI821), or low dose CBPi+low dose BETi. After 3 weeks, colonies were stained with MTT overnight and imaged.

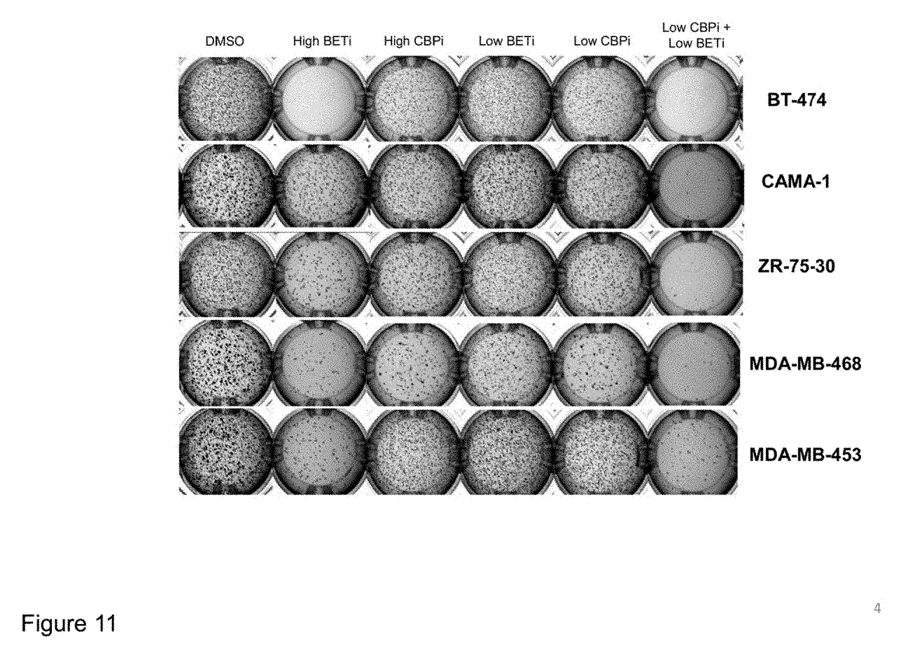

FIG. 12. Combination of CBP/EP300 and BET bromodomain inhibition has synergistic effects on the magnitude of the transcriptional response. A, Heat map showing genes modulated at least 2-fold by low dose CBPi (1.2 μM CPI778), low dose BETi (0.05 μM CPI203), or low dose CBPi+low dose BETi following a 6 h treatment in AMO-1 cells. B, Number of genes up- or downregulated at least 2-fold by the indicated treatments from A.

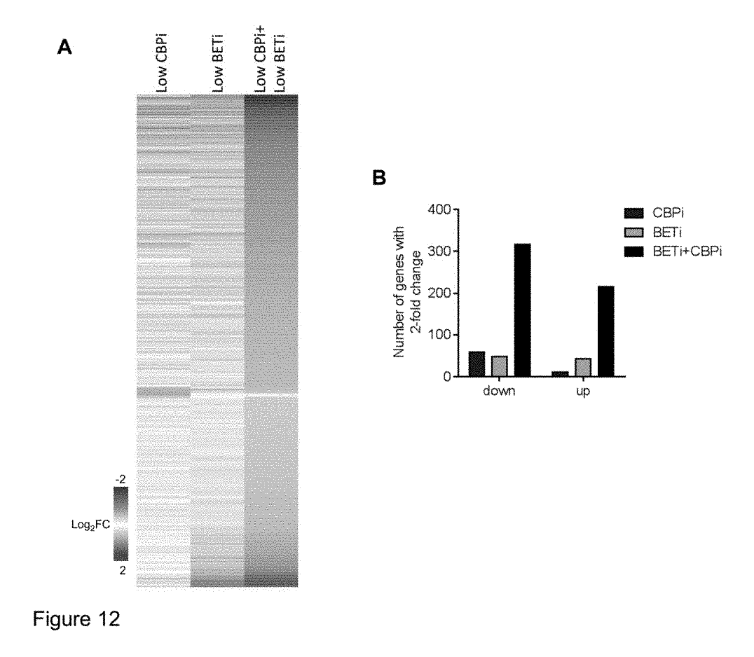

FIG. 13. Combined CBP/EP300 and BET bromodomain inhibitor treatment has distinct transcriptional effects. A, Heat map of genes modulated at least two fold by the treatments as in FIG. 5A, or with high dose CBPi (6 μM CPI778) or high dose BETi (0.25 μM CPI203). B, Venn diagrams of genes downregulated (top) or upregulated (bottom) by the indicated treatments. C, Percentage of genes up or downregulated by treatment with low CBPi+low BETi that are also regulated by high dose BETi or high dose CBPi.

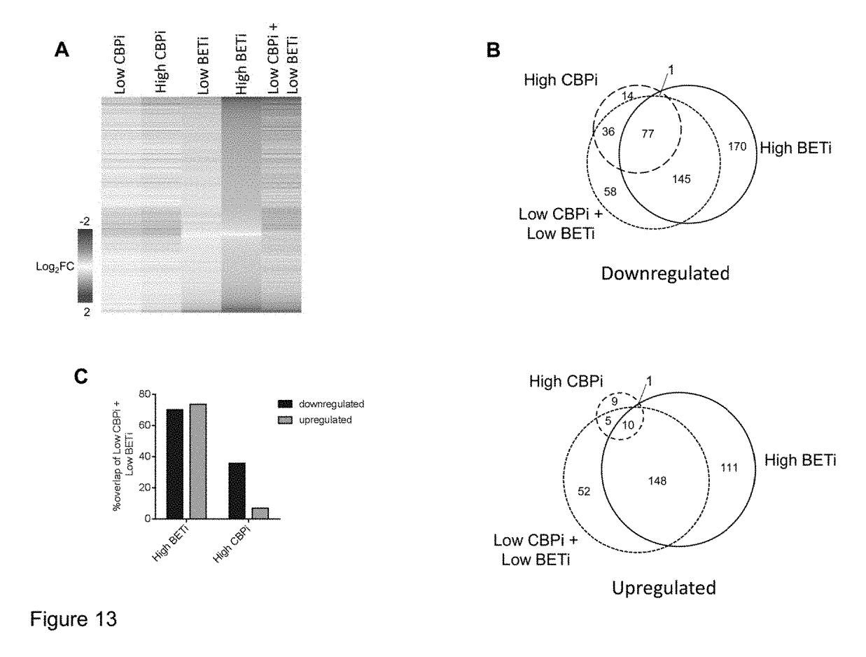

FIG. 14. A, Table of genes that show synergistic expression changes upon treatment with low dose CBPi and low dose BETi as described in FIG. 5. B, Graph showing relative expression of MYC mRNA upon the indicated treatments. C, Genes regulated at least 1.5 fold upon treatment with low dose CBPi+low dose BETi, and not significantly differentially expressed by treatment with high dose CBPi or high dose BETi.

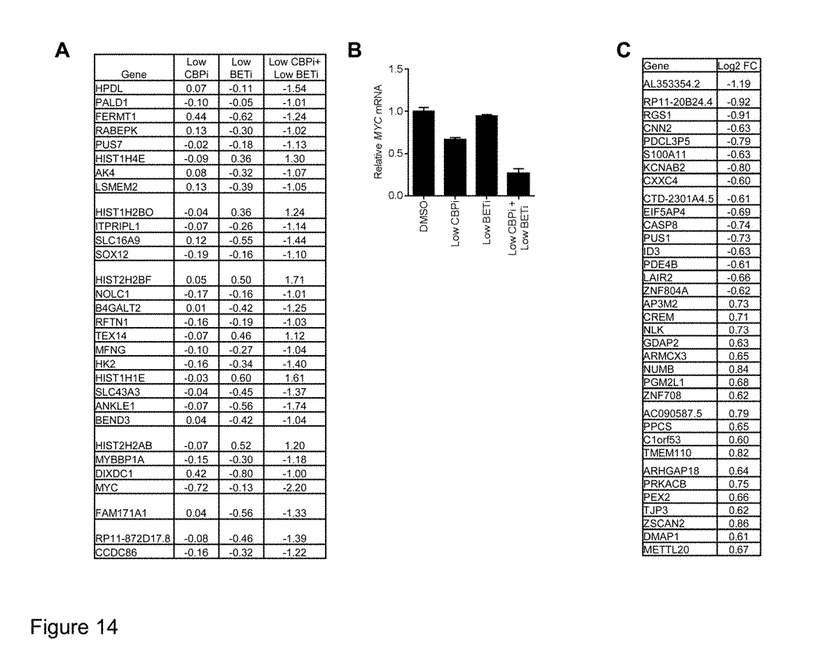

FIG. 15. A, Synergistic induction of apoptosis as measured by flow cytometric determination of sub-GI DNA content in NCI-H929 cells treated for 6 d with BETi (CPI203, 0.05 μM) and CBPi (CPI778, 1.2 μM). B, Enhanced efficacy through combined inhibition of CBP/EP300 and BET bromodomains in vivo. NCI-H929 cells were inoculated subcutaneously into female NOD-SCID mice. After tumors were palpable, mice were treated for 19 d with vehicle (methylcellulose), 0.3 mpk PO CPI821 BID (CBPi), 0.5 mpk PO CPI456 BID (BETi), or 0.5 mpk PO CPI456 BID+0.3 mpk PO CPI821 BID (BETi+CBPi). Tumor growth is expressed as a percentage of tumor size at the start of dosing. C, Quantification of tumor growth at 19 d. P-values were calculated by two-tailed unpaired t-test. D, Enhanced suppression of MYC mRNA in tumors through combined inhibition of CBP/EP300 and BET bromodomains. Tumor samples were collected 4 h after the last dose of the experiment described in B and total mRNA was isolated and used for q-RTPCR for MYC. GAPDH was used for normalization. Values are the mean and SEM of the four mice shown in each arm. P-value was calculated by two-tailed unpaired t-test.

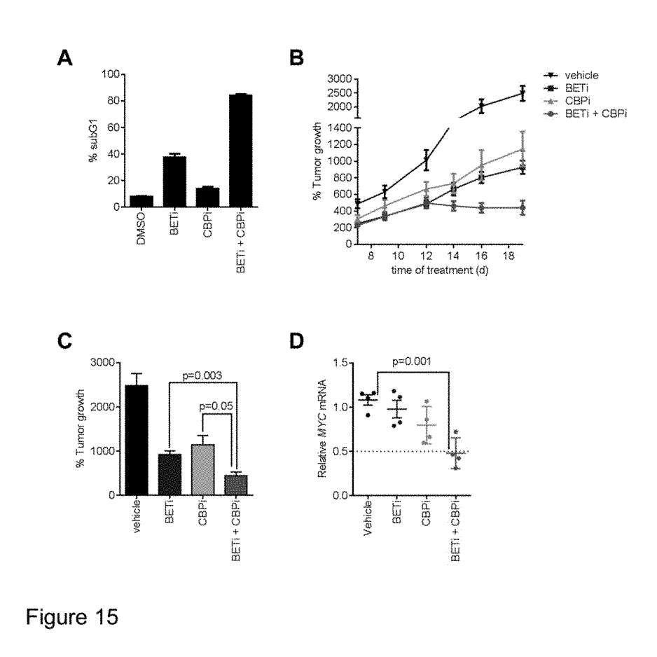

### DETAILED DESCRIPTION

The present invention is concerned with methods of treating and/or delaying progression of cancer by pharmacologically interfering with one or more of the following proteins, CBP and/or EP300, also described herein as CBP/EP300, and pharmacologically interfering with a BET protein. As such, certain embodiments of the invention provide a CBP/EP300 inhibitor in combination with a BET inhibitor for use in the prophylactic or therapeutic treatment of cancer.

### Definitions

As used herein, the term “CBP/EP300 inhibitor” refers to a compound that binds to the CBP and/or EP300 and inhibits and/or reduces a biological activity of CBP and/or EP300. In some embodiments, CBP/EP300 inhibitor substantially or completely inhibits the biological activity of the CBP and/or EP300. In some embodiments, the biological activity is binding of the CBP and/or EP300 to chromatin (e.g., histones associated with DNA) and/or another acetylated protein. In some embodiments, the biological activity is histone acetylation by CBP and/or EP300. In certain embodiments, an inhibitor has an IC₅₀ or binding constant of less about 50 μM, less than about 1 μM, less than about 500 nM, less than about 100 nM, or less than about 10 nM. In some embodiments, the CBP/EP300 inhibitor binds to and inhibits CBP bromodomain and/or CBP HAT domain. In some embodiments, the CBP/EP300 inhibitor binds to and inhibits EP300 bromodomain and/or EP300 HAT domain.

As used herein, the term “CBP/EP300 bromodomain inhibitor” refers to a compound that binds to the CBP bromodomain and/or EP300 bromodomain and inhibits and/or reduces a biological activity of CBP and/or EP300. In some embodiments, CBP/EP300 bromodomain inhibitor binds to the CBP and/or EP300 primarily (e.g., solely) through contacts and/or interactions with the CBP bromodomain and/or EP300 bromodomain. In some embodiments, CBP/EP300 bromodomain inhibitor binds to the CBP and/or EP300 through contacts and/or interactions with the CBP bromodomain and/or EP300 bromodomain as well as additional CBP and/or EP300 residues and/or domains. In some embodiments, CBP/EP300 bromodomain inhibitor substantially or completely inhibits the biological activity of the CBP and/or EP300. In some embodiments, the biological activity is binding of the bromodomain of CBP and/or EP300 to chromatin (e.g., histones associated with DNA) and/or another acetylated protein. In certain embodiments, an inhibitor has an IC₅₀ or binding constant of less about 50 μM, less than about 1 μM, less than about 500 nM, less than about 100 nM, or less than about 10 nM. In some embodiments, the CBP/EP300 bromodomain inhibitor blocks CBP/EP300 activity so as to restore a functional response by T-cells (e.g., proliferation, cytokine production, target cell killing) from a dysfunctional state to antigen stimulation. In some embodiments, the CBP/EP300 bromodomain inhibitor binds to and inhibits CBP bromodomain. In some embodiments, the CBP/EP300 bromodomain inhibitor binds to and inhibits EP300 bromodomain.

As used herein, the term “CBP/EP300 histone acetyltransferase (HAT) inhibitor” or “CBP/EP300 HAT inhibitor” refers to a compound that binds to the CBP HAT domain and/or EP300 HAT domain and inhibits and/or reduces a biological activity of CBP and/or EP300. In some embodiments, CBP/EP300 HAT inhibitor binds to the CBP and/or EP300 primarily (e.g., solely) through contacts and/or interactions with the CBP HAT domain and/or EP300 HAT domain. In some embodiments, CBP/EP300 HAT inhibitor binds to the CBP and/or EP300 through contacts and/or interactions with the CBP HAT domain and/or EP300 HAT domain as well as additional CBP and/or EP300 residues and/or domains. In some embodiments, CBP/EP300 HAT domain inhibitor substantially or completely inhibits the biological activity of the CBP and/or EP300. In some embodiments, the biological activity is binding of the HAT domain of CBP and/or EP300 to chromatin (e.g., histones associated with DNA) and/or another acetylated protein. In certain embodiments, an inhibitor has an IC₅₀ or binding constant of less about 50 μM, less than about 1μM, less than about 500 nM, less than about 100 nM, or less than about 10 nM. In some embodiments, the CBP/EP300 HAT domain inhibitor binds to and inhibits CBP HAT domain. In some embodiments, the CBP/EP300 bromodomain inhibitor binds to and inhibits EP300 HAT domain.

The terms “CBP” and “CREB binding protein,” as used herein, refers to any native CBP from any vertebrate source, including mammals such as primates (e.g. humans) and rodents (e.g., mice and rats), unless otherwise indicated. The term encompasses “full-length,” unprocessed CBP as well as any form of CBP that results from processing in the cell. The term also encompasses naturally occurring variants of CBP, e.g., splice variants or allelic variants. In some embodiments, the amino acid sequence of an exemplary human CBP is UNIPROT Q92793-1. In some embodiments, the amino acid sequence of an exemplary human CBP is UNIPROT Q92793-2. In some embodiments, the amino acid sequence of an exemplary human CBP is shown in SEQ ID NO:1.

The terms “EP300” and “E1A binding protein p300,” as used herein, refers to any native EP300 from any vertebrate source, including mammals such as primates (e.g. humans) and rodents (e.g., mice and rats), unless otherwise indicated. The term encompasses “full-length,” unprocessed EP300 as well as any form of EP300 that results from processing in the cell. The term also encompasses naturally occurring variants of EP300, e.g., splice variants or allelic variants. In some embodiments, the amino acid sequence of an exemplary human EP300 is UNIPROT Q09472. In some embodiments, the amino acid sequence of an exemplary human EP300 is shown in SEQ ID NO:2.

As used herein, the term “BET inhibitor” refers to a compound that binds to BET and inhibits and/or reduces a biological activity of BET. In some embodiments, BET inhibitor substantially or completely inhibits the biological activity of BET. In some embodiments, the biological activity is binding of BET to chromatin (e.g., histones associated with DNA) and/or another acetylated protein. In certain embodiments, a BET inhibitor has an IC₅₀ or binding constant of less about 50 μM, less than about 1 μM, less than about 500 nM, less than about 100 nM, or less than about 10 nM. In some embodiments, the BET inhibitor inhibits one or more of BRD2, BRD3, BRD4, and BRDT.

The term “Bromodomain and Extra Terminal Domain” or “BET” as used herein, refers to any native BET from any vertebrate source, including mammals such as primates (e.g. humans) and rodents (e.g., mice and rats), unless otherwise indicated. The term “BET” refers to members of the BET family, including BRD2, BRD3, BRD4, and BRDT. The term encompasses “full-length,” unprocessed BET as well as any form of BET that results from processing in the cell. The term also encompasses naturally occurring variants of BET, e.g., splice variants or allelic variants.

The terms “measurable affinity” and “measurably inhibit,” as used herein, refer to a measurable reduction in activity of a bromodomain between: (i) a sample comprising a CBP/EP300 inhibitor or composition thereof, and (ii) an equivalent sample, in the absence of said compound, or composition thereof.

“Pharmaceutically acceptable salts” include both acid and base addition salts. It is to be understood that when a compound or Example herein is shown as a specific salt, the corresponding free-base, as well as other salts of the corresponding free-base (including pharmaceutically acceptable salts of the corresponding free-base) are contemplated.

“Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particular organic non-toxic bases are isopropylamine, diethylamine, ethanolamine, tromethamine, dicyclohexylamine, choline, and caffeine.

A “solvate” refers to an association or complex of one or more solvent molecules and a compound of the present invention. Examples of solvents include water, isopropanol, ethanol, methanol, DMSO, ethyl acetate, acetic acid and ethanolamine. The term “hydrate” refers to the complex where the solvent molecule is water.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The phrase “substantially similar,” as used herein, refers to a sufficiently high degree of similarity between two numeric values (generally one associated with a molecule and the other associated with a reference/comparator molecule) such that one of skill in the art would consider the difference between the two values to not be of statistical significance within the context of the biological characteristic measured by said values (e.g., Kd values). The difference between said two values may be, for example, less than about 20%, less than about 10%, and/or less than about 5% as a function of the reference/comparator value. The phrase “substantially normal” refers to substantially similar to a reference (e.g., normal reference).

The phrase “substantially different,” refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values may be, for example, greater than about 10%, greater than about 20%, greater than about 30%, greater than about 40%, and/or greater than about 50% as a function of the value for the reference/comparator molecule.

An ‘effective amount’ of an agent, e.g., a pharmaceutical formulation, refers to an amount effective, at dosages and for periods of time necessary, to achieve the desired therapeutic or prophylactic result. In some embodiments, the effective amount refers to an amount of a CBP/EP300 and/or BET inhibitor that (i) treats the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. In some embodiments, the effective amount of the CBP/EP300 and/or BET inhibitor may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. For cancer therapy, efficacy can, for example, be measured by assessing the time to disease progression (TTP) and/or determining the response rate (RR). In some embodiments, an effective amount is an amount of a chemical entity described herein sufficient to significantly decrease the activity or number of drug tolerant or drug tolerant persisting cancer cells.

“Treatment” (and variations such as “treat” or “treating”) refers to clinical intervention in an attempt to alter the natural course of the individual or cell being treated, and can be performed either for prophylaxis or during the course of clinical pathology. Desirable effects of treatment include one or more of preventing occurrence or recurrence of disease, alleviation of symptoms, diminishment of any direct or indirect pathological consequences of the disease, stabilized (i.e., not worsening) state of disease, preventing metastasis, decreasing the rate of disease progression, amelioration or palliation of the disease state, prolonging survival as compared to expected survival if not receiving treatment and remission or improved prognosis. In certain embodiments, a CBP/EP300 inhibitor and BET inhibitor are used to delay development of a disease or disorder or to slow the progression of a disease or disorder. Those individuals in need of treatment include those already with the condition or disorder as well as those prone to have the condition or disorder, (for example, through a genetic mutation or aberrant expression of a gene or protein) or those in which the condition or disorder is to be prevented.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

The term “patient” or “individual” as used herein, refers to an animal, such as a mammal, such as a human. In one embodiment, patient or individual refers to a human.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

In one embodiment the cytotoxic agent is selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one embodiment the cytotoxic agent is a taxane. In one embodiment the taxane is paclitaxel or docetaxel. In one embodiment the cytotoxic agent is a platinum agent. In one embodiment the cytotoxic agent is an antagonist of EGFR. In one embodiment the antagonist of EGFR is N-(3-ethynylphenyl)-6,7-bis(2-methoxyethoxy)quinazolin-4-amine (e.g., erlotinib). In one embodiment the cytotoxic agent is a RAF inhibitor. In one embodiment, the RAF inhibitor is a BRAF and/or CRAF inhibitor. In one embodiment the RAF inhibitor is vemurafenib. In one embodiment the cytotoxic agent is a PI3K inhibitor.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁ λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943, 533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18- OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugswith analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

As is understood by one skilled in the art, reference to “about” a value or parameter herein includes (and describes) embodiments that are directed to that value or parameter per se. For example, description referring to “about X” includes description of “X”.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. It is understood that aspect and embodiments of the invention described herein include “consisting” and/or “consisting essentially of” aspects and embodiments.

Recitation of ranges of values herein are merely intended to serve as a shorthand method of referring individually to each separate value falling within the range, unless otherwise indicated herein, and each separate value is incorporated into the specification as if it were individually recited herein.

### Uses of CBP/EP300 and BET Inhibitors

Provided herein are methods of using a CBP/EP300 inhibitor for the inhibition of a CBP/EP300 bromodomain and/or CBP/EP300 HAT domain and a BET inhibitor for the inhibition of BET (in vitro or in vivo). For example, provided herein are methods for treating a CBP/EP300 bromodomain-mediated, a CBP/EP300 HAT domain-mediated, and/or a BET-mediated disorder in an individual comprising administering a CBP/EP300 inhibitor to the individual in combination with a BET inhibitor. In some embodiments, the bromodomain-mediated, HAT domain-mediated disorder, and/or BET-mediated disorder is cancer.

Provided herein are methods for treating or delaying progression of cancer in an individual comprising administering to the individual an effective amount of a CBP/EP300 inhibitor in combination with a BET inhibitor. In some embodiments, the CBP/EP300 inhibitor binds to a bromodomain of CBP/EP300. In some embodiments, the CBP/EP300 inhibitor binds to one or more residues of the amino acid sequence KKIFKPEELRQALMPTLEALYRQDPESLPFRQPVDPQLLGIPDYFDIVKNPMDLST IKRKLDTGQYQEPWQYVDDVWLMFNNAWLYNRKTSRVYKFCSKLAEVFEQEIDPVMQ SLG (amino acid residues 1082-1197 of UniProt No. Q92793 (SEQ ID NO:5)). In some embodiments, the CBP/EP300 inhibitor binds to one or more residues of the amino acid sequence RQDPESLPFRQPVDPQLLGIPDYFDIVKONPMDLSTIKRKLDTGQYQEPWQYVDDVWLMF NNAWLYNRKTSRVY (amino acid residues 1103-1175 of UniProt No. Q92793 (SEQ ID NO:3)). In some embodiments, the CBP/EP300 inhibitor binds to a bromodomain of EP300. In some embodiments, the CBP/EP300 inhibitor binds to one or more residues of the amino acid sequence APGQSKKKIFKPEELRQALMPTLEALYRQDPESLPFRQPVDPQLLGIPDYFDIVKSPMD LSTIKRKLDTGQYQEPWQYVDDIWLMFNNAWLYNRKTSRVYKYCSKLSEVFEQEIDPV MQSLG (amino acid residues 1040-1161 of UniProt No. Q09472 (SEQ ID NO:6)). In some embodiments, the CBP/EP300 inhibitor binds to one or more residues of the amino acid sequence RQDPESLPFRQPVDPQLLGIPDYFDIVKSPMDLSTIKRKLDTGQYQEPWQYVDDIWLMFN NAWLYNRKTSRVY (amino acid residues 1067-1139 of UniProt No. Q09472 (SEQ ID NO:4)). In some embodiments, the CBP/EP300 inhibitor binds to the bromodomain of EP300 and the bromodomain of CBP. In some embodiments, the CBP/EP300 inhibitor binds SEQ ID NO:5 and SEQ ID NO:6. In some embodiments, the CBP/EP300 inhibitor binds SEQ ID NO:3 and SEQ ID NO:4. In some embodiments, the CBP/EP300 inhibitor inhibits and/or reduces binding of the CBP/EP300 bromodomain to chromatin.

In some embodiments, the CBP/EP300 inhibitor binds to a HAT domain of CBP/EP300.

### i. Combinations of a CBP/EP300 Inhibitor and a BET Inhibitor

Another embodiment includes a method of treating cancer in an individual comprising administering to the individual (a) a CBP/EP300 inhibitor and (b) a BET inhibitor. Further provided herein methods of extending the duration of response in an individual with cancer comprising administering to the individual (a) an effective amount of a CBP/EP300 inhibitor and (b) an effective amount of a BET inhibitor. In some embodiments, the CBP/EP300 inhibitor and the BET inhibitor are concomitantly administered. In certain embodiments, the CBP/EP300 inhibitor is administered prior to and/or concurrently with the BET inhibitor. In some embodiments, the CBP/EP300 inhibitor and the BET inhibitor are co-administered. In some embodiments, the CBP/EP300 inhibitor and the BET inhibitor are co-formulated. In some embodiments, the CBP/EP300 inhibitor is administered separately from the BET inhibitor. In some embodiments, the CBP/EP300 inhibitor is administered sequentially with the BET inhibitor. In some embodiments, the CBP/EP300 inhibitor is administered simultaneously with the BET inhibitor. In some embodiments, the individual is administered the BET inhibitor and subsequently administered the CBP/EP300 inhibitor. In some embodiments, the individual is administered the CBP/EP300 inhibitor and subsequently administered the BET inhibitor.

In some embodiments, the administration of the CBP/EP300 inhibitor and BET inhibitor delays development of resistance. In some embodiments, the administration of the CBP/EP300 inhibitor and BET inhibitor provides a longer duration of response. For example, the duration of response may be increased 1-fold, 2-fold, 3-fold, 4-fold, 5-fold, or 10-fold.

### ii. Use of CBP/EP300 Inhibitor in Treatment of BET Inhibitor Resistant Cancers

In some embodiments, the CBP/EP300 inhibitor is used to treat cancers where the cancer is resistant to BET inhibitors. In some embodiments, BET resistant cells demonstrate half-maximal growth inhibition at a concentration of BET inhibitor that is greater than 10-fold that of parental cells. In some embodiments, parental cells treated with the BET inhibitor undergo apoptosis, a phenomenon that correlates with strong suppression of MYC and the anti-apoptotic gene, BCL2. In some embodiments, transcription of MYC and BCL2 in BET inhibitor resistant cells is maintained in the presence of the BET inhibitor, and the apoptotic effect is severely blunted. In some embodiments, withdrawal of the BET inhibitor from the resistant cells triggers apoptosis. In some embodiments, MYC transcription in BET inhibitor resistant cells remained dependent on acetyl-lysine signaling and the bromodomains of CBP/EP300. In some embodiments a CBP/EP300 inhibitor transcriptionally silences MYC expression in myeloma and leukemia derived cell lines.

### iii. Disorders

Examples of CBP/EP300 bromodomain-mediated, CBP/EP300 HAT domain-mediated, and/or BET-mediated disorders include cancers, including, but not limited, to acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cellleukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies off-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In certain embodiments, the cancer is a B-cell proliferative cancer. In certain embodiments, the cancer is leukemia or lymphoma. In certain embodiments, the cancer is leukemia. In certain embodiments, the cancer is myeloma. In certain embodiments, the cancer is breast cancer.

The amount of both the CBP/EP300 inhibitor or salt thereof and BET inhibitor (in those compositions which comprise an additional therapeutic agent as described above) that may be combined with the carrier materials to produce a single dosage form or separate dosage forms will vary depending upon the host treated and the particular mode of administration. In certain embodiments, compositions of this invention are formulated such that a dosage of between 0.01-100 mg/kg body weight/day of the CBP/EP300 inhibitor and/or BET inhibitor can be administered.

The CBP/EP300 and BET inhibitor may act synergistically. In some embodiments, the combination of CBP/EP300 and BET inhibitor may slow the growth of cancer cells 5-fold, 10-fold, 50-fold or 100-fold over the administration of either inhibitor alone. Therefore, the amount of one therapeutic agent in such compositions may be less than that required in a monotherapy utilizing only that therapeutic agent, or there may be fewer side effects for the patient given that a lower dose is used. In certain embodiments, in such compositions a dosage of between 0.01-1,000 μg/kg body weight/day of the additional therapeutic agent can be administered.

### CBP/EP300 Inhibitors and BET Inhibitors

It has been discovered that certain compounds are CBP/EP300 inhibitors that bind specifically to the bromodomain motifs harbored in one or more of CBP and/or EP300 and other certain compounds are CBP/EP300 inhibitors that bind specifically to the HAT domain motifs harbored in one or more of CBP and/or EP300.

In some embodiments, the CBP/EP300 inhibitor binds to a bromodomain of CBP. In some embodiments, the CBP/EP300 inhibitor binds to one or more residues of the amino acid sequence of SEQ ID NO:5. In some embodiments, the CBP/EP300 inhibitor binds to one or more residues of the amino acid sequence of SEQ ID NO:3. In some embodiments, the CBP/EP300 inhibitor binds to a bromodomain of EP300. In some embodiments, the CBP/EP300 inhibitor binds to one or more residues of the amino acid sequence of SEQ ID NO:6. In some embodiments, the CBP/EP300 inhibitor binds to one or more residues of the amino acid sequence of SEQ ID NO:4. In some embodiments, the CBP/EP300 inhibitor binds to the bromodomain of EP300 and the bromodomain of CBP. In some embodiments, the CBP/EP300 inhibitor binds SEQ ID NO:5 and SEQ ID NO:6. In some embodiments, the CBP/EP300 inhibitor binds SEQ ID NO:3 and SEQ ID NO:4. In some embodiments, the CBP/EP300 inhibitor binds to at least one (e.g., 1, 2, 3, 4, 5, 6, 7, 8, 9, 10, 11, 12 or 13) of the following CBP residues: LEU 1109, PRO 1110, PHE 1111, VAL 1115, LEU 1120, ILE 1122, TYR 1125, ALA 1164, TYR 1167, ASN 1168, ARG 1173, VAL 1174 or PHE 1177. In some embodiments, the CBP/EP300 inhibitor binds to at least one (e.g., 1, 2, 3, 4, 5, 6, 7, 8, 9, 10, 11, 12 or 13) of the following EP300 residues: LEU 1073, PRO 1074, PHE 1075, VAL 1079, LEU 1084, ILE 1086, TYR 1089, ALA 1128, TYR 1131, ASN 1132, ARG 1137, VAL 1138 or TYR 1141.

In some embodiments, the CBP/EP300 inhibitor interferes with the associating of CBP and/or EP300 with histones, in particular acetylated lysines in histones. In some embodiments, the CBP/EP300 inhibitor inhibits binding of CBP and/or EP300 to chromatin (e.g., histone associated DNA). In some embodiments, the CBP/EP300 inhibitor inhibits and/or reduces binding of the CBP bromodomain and/or EP300 bromodomain to chromatin (e.g., histone associated DNA). In some embodiments, the CBP/EP300 inhibitor does not affect association of other domains of CBP and/or EP300 to chromatin. In some embodiments, CBP/EP300 inhibitor binds to the CBP and/or EP300 primarily (e.g., solely) through contacts and/or interactions with the CBP bromodomain and/or EP300 bromodomain. In some embodiments, CBP/EP300 inhibitor binds to the CBP and/or EP300 through contacts and/or interactions with the CBP bromodomain and/or EP300 bromodomain as well as additional CBP and/or EP300 residues and/or domains. Methods of assaying association with chromatin are known in the art and include, but are not limited to, chromatin fractionation, BRET assay (Promega), FRAP assay, Chromatin Immunoprecipitation (ChIP), biophysical binding assay, and/or Histone Association Assay. See, e.g., Das et al., BioTechniques 37:961-969 (2004).

In some embodiments, the CBP/EP300 inhibitor binds to the HAT domain of CBP and/or EP300. In some embodiments, the CBP/EP300 inhibitor binds to the HAT domain of CBP and/or EP300 as identified in Delvecchio et al., Nat. Struct. & Mol. Biol. 20:1040-1046 (2013), which is incorporated by reference in its entirety. In some embodiments, the CBP/EP300 inhibitor substantially binds to one or more residues of the amino acid sequence ENKFSAKRLQTTR LGNHLEDRVNKFLRRQNHPEAGEVFVRVVASSDKTVEVKPGMKSRFVDSGEMSESFPY RTKALFAFEEIDGVDVCFFGMHVQEYGSDCPPPNTRRVYISYLDSIHFFRPRCLRTAVYH EILIGYLEYVKKLGYVTGHIWACPPSEGDDYIFHCHPPDQKIPKPKRLQEWYKKIVILDKAF AERIIHDYKDIFKQATEDRLTSAKELPYFEGDFWPNVLEESIKELEQEEEERKKEESTAAS ETTEGSQGDSKNAKKKNNKKTNKNKSSISRANKKKPSMPNVSNDLSQKLYATMEKHKE VFFVIHLHAGPVINTLPPIVDPDPLLSCDLMDGRDAFLTLARDKHWEFSSLRRSKWSTLC MLVELHTQGQD (amino acid residues 1321-1701 of UniProt No. Q92793 (SEQ ID NO:8)). In some embodiments, the CBP/EP300 bromodomain inhibitor substantially binds to one or more residues of the amino acid sequence ENKFSAKRLPSTRLGTFLENRVNDFLRRQNHPESGEVTVRVVHASDKTVEVKPGMKARF VDSGEMAESFPYRTKALFAFEEIDGVDLCFFGMHVQEYGSDCPPPNQRRVYISYLDSVHF FRPKCLRTAVYHEILIGYLEYVKKLGYTTGHIWACPPSEGDDYIFHCHPPDQKIPKPKRLQ EWYKKMLDKAVSERIVHDYKDIFKQATEDRLTSAKELPYFEGDFWPNVLEESIKELEQE EEERKREENTSNESTDVTKGDSKNAKKKNNKKTSKNKSSLSRGNKKKPGMPNVSNDLS QKLYATMEKHKEVFFVIRLIAGPAANSLPPIVDPDPLIPCDLMDGRDAFLTLARDKHLEFS SLRRAQWSTMCMLVELHTQSQD (amino acid residues 1285-1664 of UniProt No. Q09472 (SEQ ID NO:7)). In some embodiments, the CBP/EP300 bromodomain inhibitor inhibits the histone acetyltransferase (HAT) catalytic activity of CBP and/or EP300.

Descriptions of CBP and EP300 (also known as p300) can be found, e.g., in Chrivia et al., Nature, 365, 855 (1993) and Teufel et al., PNAS, 104, 7009 (2007). Certain compounds that may be used as CBP/EP300 inhibitors include the following compounds:

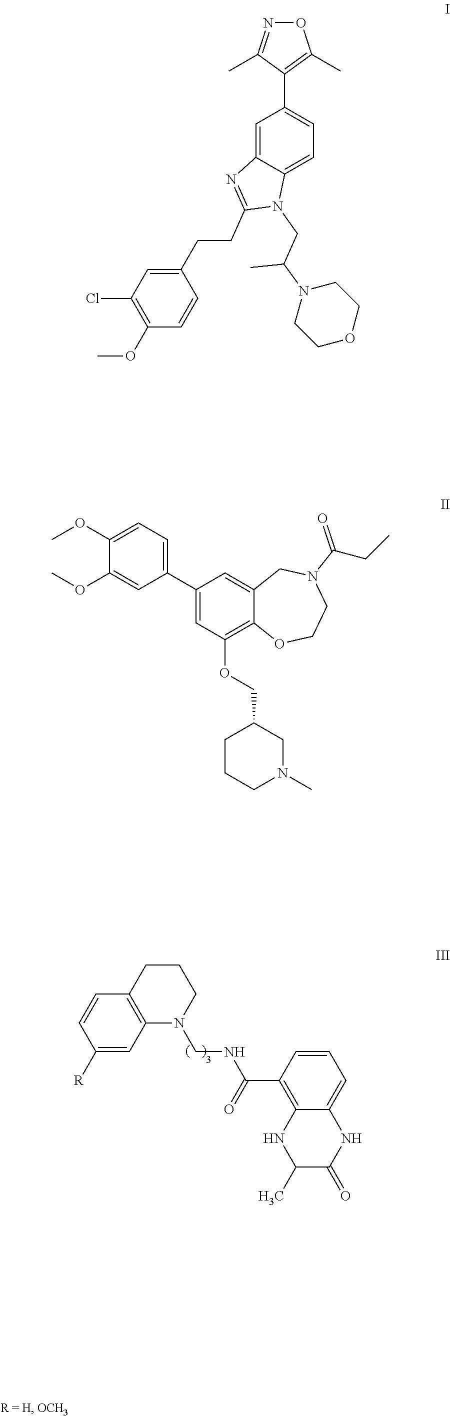

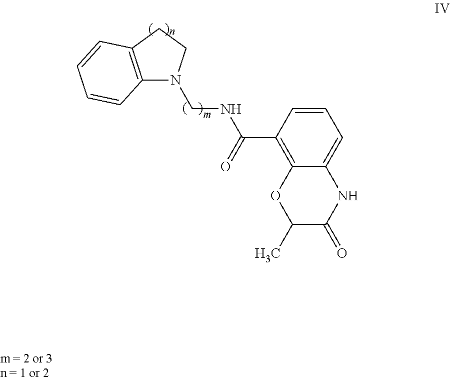

### Pharmaceutical Compositions and Methods of Administration

Further provided herein are pharmaceutical compositions comprising a CBP/EP300 inhibitor, pharmaceutical compositions comprising a BET inhibitor, or pharmaceutical compositions comprising co-formulated CBP/EP300 and BET inhibitors, or salts thereof for use in the methods described herein. In one embodiment, the composition further comprises a pharmaceutically acceptable carrier, adjuvant, or vehicle. In another embodiment, the composition further comprises an amount of the compound effective to measurably inhibit a CBP/EP300 bromodomain, CBP/EP300 HAT domain, and/or BET. In certain embodiments, the composition is formulated for administration to a patient in need thereof.

Compositions comprising a CBP/EP300 inhibitor, or a BET inhibitor, or co-formulated CBP/EP300 and BET inhibitors, or salts thereof, may be administered orally, parenterally, by inhalation spray, topically, transdermally, rectally, nasally, buccally, sublingually, vaginally, intraperitoneal, intrapulmonary, intradermal, epidural or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In one embodiment, the composition comprising a CBP/EP300 inhibitor, or a BET inhibitor, or co-formulated CBP/EP300 and BET inhibitors, or salts thereof, is formulated as a solid dosage form for oral administration. Solid dosage forms for oral administration include capsules, tablets, pills, powders, and granules. In certain embodiments, the solid oral dosage form comprising a CBP/EP300 inhibitor, or a BET inhibitor, or co-formulated CBP/EP300 and BET inhibitors, or salts thereof, further comprises one or more of (i) an inert, pharmaceutically acceptable excipient or carrier, such as sodium citrate or dicalcium phosphate, and (ii) filler or extender such as starches, lactose, sucrose, glucose, mannitol, or silicic acid, (iii) binders such as carboxymethylcellulose, alginates, gelatin, polyvinylpyrrolidinone, sucrose or acacia, (iv) humectants such as glycerol, (v) disintegrating agent such as agar, calcium carbonate, potato or tapioca starch, alginic acid, certain silicates or sodium carbonate, (vi) solution retarding agents such as paraffin, (vii) absorption accelerators such as quaternary ammonium salts, (viii) a wetting agent such as cetyl alcohol or glycerol monostearate, (ix) absorbent such as kaolin or bentonite clay, and (x) lubricant such as talc, calcium stearate, magnesium stearate, polyethylene glycols or sodium lauryl sulfate. In certain embodiments, the solid oral dosage form is formulated as capsules, tablets or pills. In certain embodiments, the solid oral dosage form further comprises buffering agents. In certain embodiments, such compositions for solid oral dosage forms may be formulated as fillers in soft and hard-filled gelatin capsules comprising one or more excipients such as lactose or milk sugar, polyethylene glycols and the like.

In certain embodiments, tablets, dragees, capsules, pills and granules of the compositions comprising a CBP/EP300 inhibitor, or a BET inhibitor, or co-formulated CBP/EP300 and BET inhibitors, or salts thereof, optionally comprise coatings or shells such as enteric coatings. They may optionally comprise opacifying agents and can also be of a composition that they release the active ingredient(s) only, or preferentially, in a certain part of the intestinal tract, optionally, in a delayed manner. Examples of embedding compositions include polymeric substances and waxes, which may also be employed as fillers in soft and hard-filled gelatin capsules using such excipients as lactose or milk sugar as well as high molecular weight polethylene glycols and the like.

In another embodiment, a composition comprises a micro-encapsulated CBP/EP300 inhibitor, or BET inhibitor, or co-formulated CBP/EP300 and BET inhibitors, or salts thereof and optionally, further comprises one or more excipients.

In another embodiment, compositions comprise liquid dosage formulations comprising a CBP/EP300 inhibitor, or a BET inhibitor, or co-formulated CBP/EP300 and BET inhibitors, or salts thereof, for oral administration, and optionally further comprise one or more of pharmaceutically acceptable emulsions, microemulsions, solutions, suspensions, syrups and elixirs. In certain embodiments, the liquid dosage form optionally, further comprise one or more of an inert diluent such as water or other solvent, a solubilizing agent, and an emulsifier such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols or fatty acid esters of sorbitan, and mixtures thereof. In certain embodiments, liquid oral compositions optionally further comprise one or more adjuvant, such as a wetting agent, a suspending agent, a sweetening agent, a flavoring agent and a perfuming agent.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a CBP/EP300 inhibitor, or a BET inhibitor, or co-formulated CBP/EP300 and BET inhibitors, or salts thereof, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In certain embodiments, the composition for rectal or vaginal administration are formulated as suppositories which can be prepared by mixing a CBP/EP300 inhibitor, or a BET inhibitor, or co-formulated CBP/EP300 and BET inhibitors, or salts thereof, with suitable non-irritating excipients or carriers such as cocoa butter, polyethylene glycol or a suppository wax, for example those which are solid at ambient temperature but liquid at body temperature and therefore melt in the rectum or vaginal cavity and release the CBP/EP300 inhibitor and/or BET inhibitor.

Example dosage forms for topical or transdermal administration of a CBP/EP300 inhibitor, or a BET inhibitor, or co-formulated CBP/EP300 and BET inhibitors, or salts thereof, include ointments, pastes, creams, lotions, gels, powders, solutions, sprays, inhalants or patches. The CBP/EP300 inhibitor, or a BET inhibitor, or co-formulated CBP/EP300 and BET inhibitors, or salts thereof, are admixed under sterile conditions with a pharmaceutically acceptable carrier, and optionally preservatives or buffers. Additional formulation examples include an ophthalmic formulation, ear drops, eye drops, transdermal patches. Transdermal dosage forms can be made by dissolving or dispensing the CBP/EP300 inhibitor, or the BET inhibitor, or co-formulated CBP/EP300 and BET inhibitors, or salts thereof, in medium, for example ethanol or dimethylsulfoxide. Absorption enhancers can also be used to increase the flux of the compound across the skin. The rate can be controlled by either providing a rate controlling membrane or by dispersing the compound in a polymer matrix or gel.

Nasal aerosol or inhalation formulations of a CBP/EP300 inhibitor, or a BET inhibitor, or co-formulated CBP/EP300 and BET inhibitors, or salts thereof, may be prepared as solutions in saline, employing benzyl alcohol or other suitable preservatives, absorption promotors to enhance bioavailability, fluorocarbons, and/or other conventional solubilizing or dispersing agents.

In certain embodiments, pharmaceutical compositions may be administered with or without food. In certain embodiments, pharmaceutically acceptable compositions are administered without food. In certain embodiments, pharmaceutically acceptable compositions of this invention are administered with food.

Specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided CBP/EP300 inhibitor, or BET inhibitor, or co-formulated CBP/EP300 and BET inhibitors, or salts thereof, in the composition will also depend upon the particular compound in the composition.

In one embodiment, the effective amount of the compound of the invention administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, contain from about 5 to about 100 mg of the compound of the invention.

An example tablet oral dosage form comprises about 2 mg, 5 mg, 25 mg, 50 mg, 100 mg, 250 mg or 500 mg of a CBP/EP300 inhibitor, or a BET inhibitor, or co-formulated CBP/EP300 and BET inhibitors, or salts thereof, and further comprises about 5-30 mg anhydrous lactose, about 5-40 mg sodium croscarmellose, about 5-30 mg polyvinylpyrrolidone (PVP) K30 and about 1-10 mg magnesium stearate. The process of formulating the tablet comprises mixing the powdered ingredients together and further mixing with a solution of the PVP. The resulting composition can be dried, granulated, mixed with the magnesium stearate and compressed to tablet form using conventional equipment. An example of an aerosol formulation can be prepared by dissolving about 2-500 mg of a compound of formula I or salt thereof, in a suitable buffer solution, e.g. a phosphate buffer, and adding a tonicifier, e.g. a salt such sodium chloride, if desired. The solution may be filtered, e.g. using a 0.2 micron filter, to remove impurities and contaminants.

The CBP/EP300 inhibitor, BET inhibitor, or co-formulated CBP/EP300 and BET inhibitors, or salts thereof, may be employed alone or in combination with other agents for. For example, the second agent of the pharmaceutical combination formulation or dosing regimen may have complementary activities to the CBP/EP300 inhibitor or BET inhibitor such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a CBP/EP300 inhibitor, or a BET inhibitor, or co-formulated CBP/EP300 and BET inhibitors, or salts thereof, and a further active pharmaceutical ingredient or ingredients, including cytotoxic agents, chemotherapeutic agents, and/or radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

### EXAMPLES

The following are examples of methods and compositions of the invention. It is understood that various other embodiments may be practiced, given the general description provided above.

### Example 1

### CBP/EP300 Inhibitors and BET Inhibitors in 6-Day Viability Assay with Leukemia and Breast Cancer Cell Lines

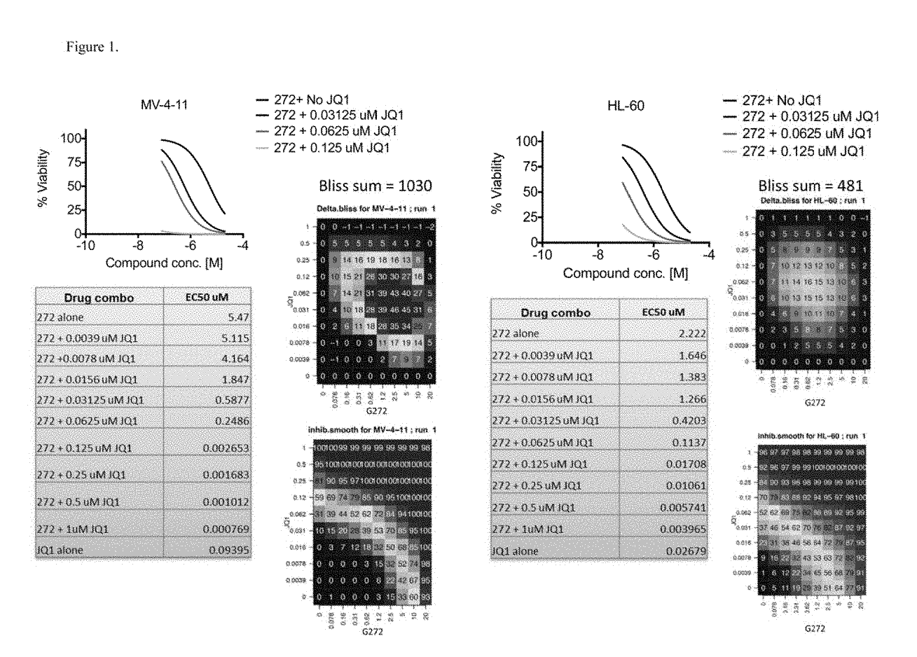

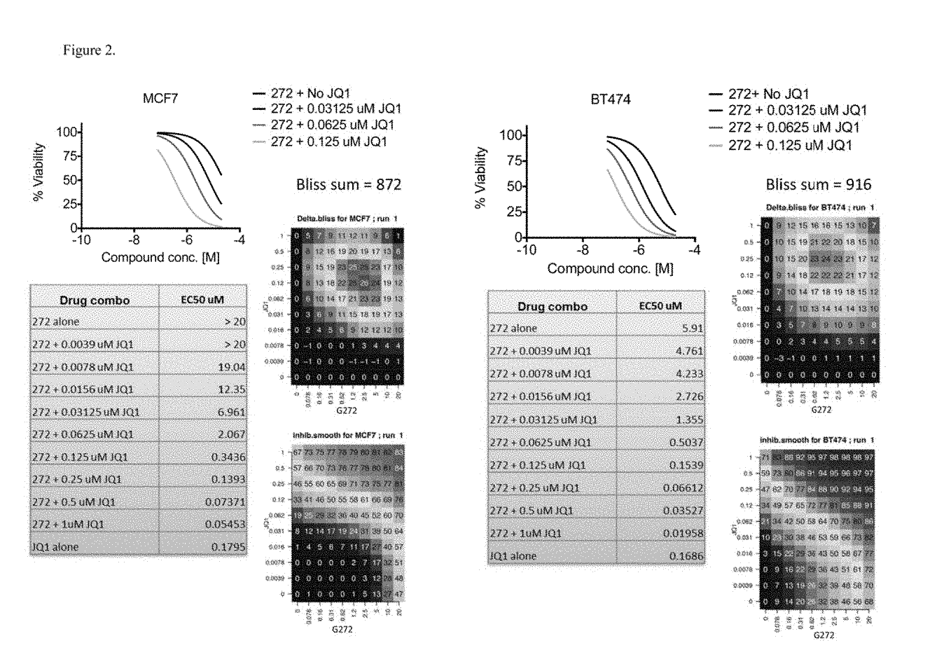

Leukemia (MV-4-11 and HL-60) and Breast Cancer (MCF7 and BT474) cell lines were plated into 384 well plates with RPMI media containing 10% FBS (fetal bovine serum) and allowed to incubate at 37° C. for 24 hours. CBP/EP300 inhibitors (G272) and BET inhibitor (JQ1) were dissolved in DMSO and added to the cells in a concentration gradient from 0 to 20 μM for CBP inhibitor (G272), and 0 to 1 μM for JQ1, and the cells were incubated at 37° C. for 6 days. After six days of treatment, percent cell viability and EC50 were determined by CellTiter-Glo. Results for leukemia cell lines are shown in FIG. 1. Results for breast cancer cell lines are shown in FIG. 2. Synergy was monitored by Bliss score.

### Example 2

### Generation of BET Inhibitor Resistant Cells

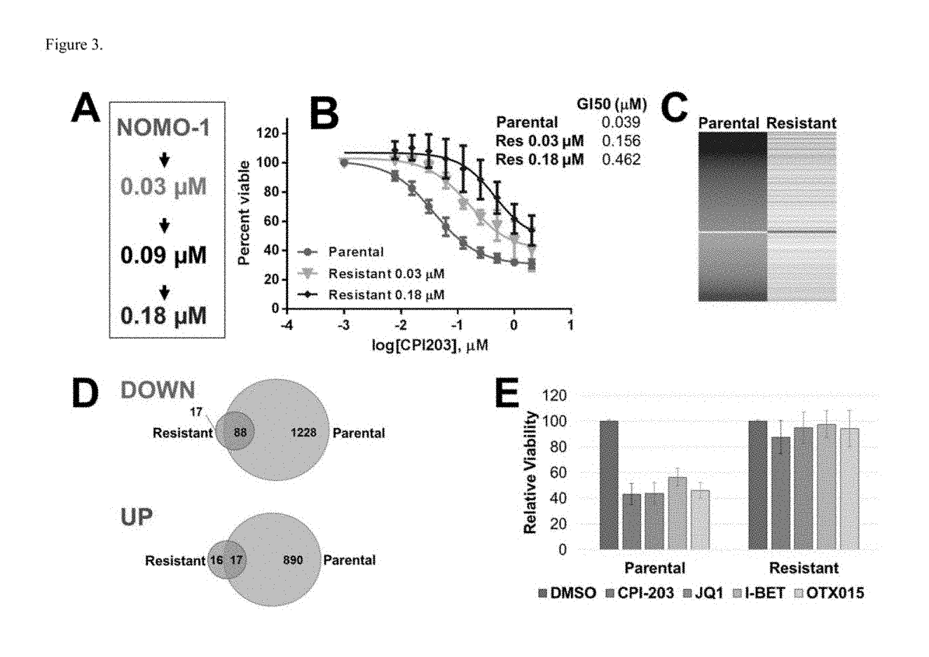

A. NOMO-1 cells were passaged in the presence of increasing concentrations of BET inhibitor (CPI203) every 8-10 weeks. B. Cells were treated with increasing concentrations of BET inhibitor (CPI203) and viability was measured after 4 days by resazurin staining. GI50 values were calculated with GraphPad Prism. C. Heat map depicting genes with ≧2-fold change in expression when challenged with high dose CPI203 (2 μM) for 24 hours relative to basal expression (Parental, DMSO; Resistant, 0.18 μM CPI203). C. Cells were treated as in (B), and Venn diagram depicts number of genes with ≧2-fold change in expression relative to basal levels. E. Cells were treated with 2 μM of each inhibitor and viability was measured after 4 days by resazurin staining. Results are shown in FIG. 3.

### Example 3

### Dysfunctional Apoptosis in BET Inhibitor Resistant Cells

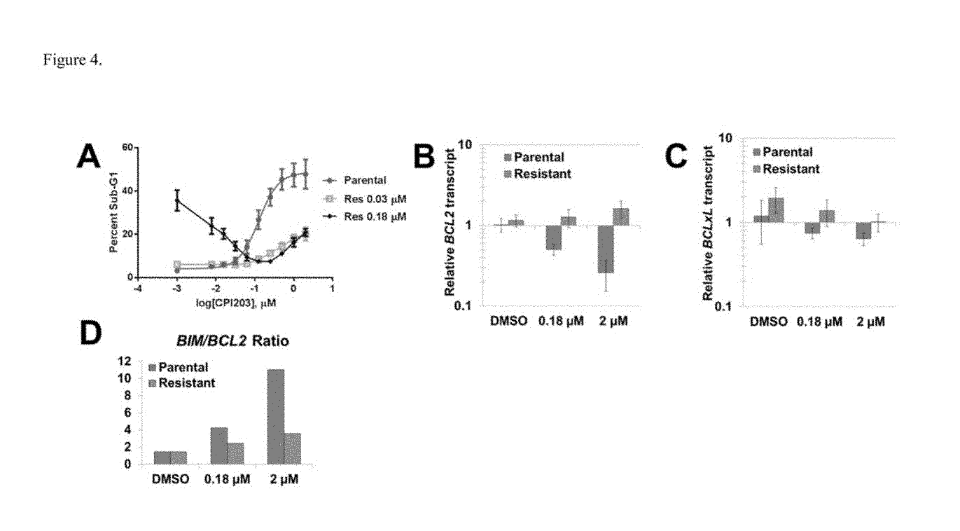

A. Cells were treated with increasing concentrations of BET inhibitor (CPI203) and the % sub-G1 (% apoptotic) was measured after 4 days by flow cytometry. B-C. Cells were treated with indicated concentrations of BET inhibitor (CPI203) for 24 hours and BCL2 (B) or BCLxL (C) mRNA was quantified by qRT-PCR. C. BIM and BCL2 transcripts were quantified by qRT-PCR, normalized to the DMSO control, and subsequently the ratio of BIM/BCL2 was calculated. Results are shown in FIG. 4.

### Example 4

### BET Inhibitor Resistant Cells Maintain MYC Expression

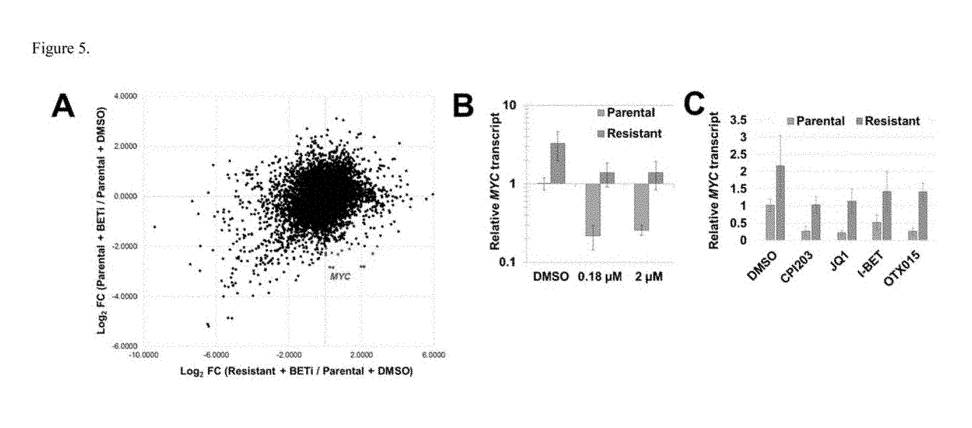

A. RNA-sequencing was performed on parental cells treated with DMSO or 0.18 μM BET inhibitor (CPI203) and resistant cells treated with 0.18 μM BET inhibitor (CPI203) for 24 hours. Log2 fold change in gene expression is plotted. Lower right-hand quadrant indicates genes that are ≧4× down-regulated by BET inhibitor (CPI203) in parental cells, but are unchanged or up-regulated in resistant cells compared to parental. B. Cells were treated with increasing concentrations of BET inhibitor (CPI203) and MYC mRNA was quantified by qRT-PCR. C. Cells were treated for 4 hours with 0.25 μM each inhibitor subsequent to analysis of MYC mRNA levels by qRT-PCR. Results are shown in FIG. 5.

### Example 5

### CBP/EP300 Bromodomains are Required for MYC Expression in BET Inhibitor Resistant Cells

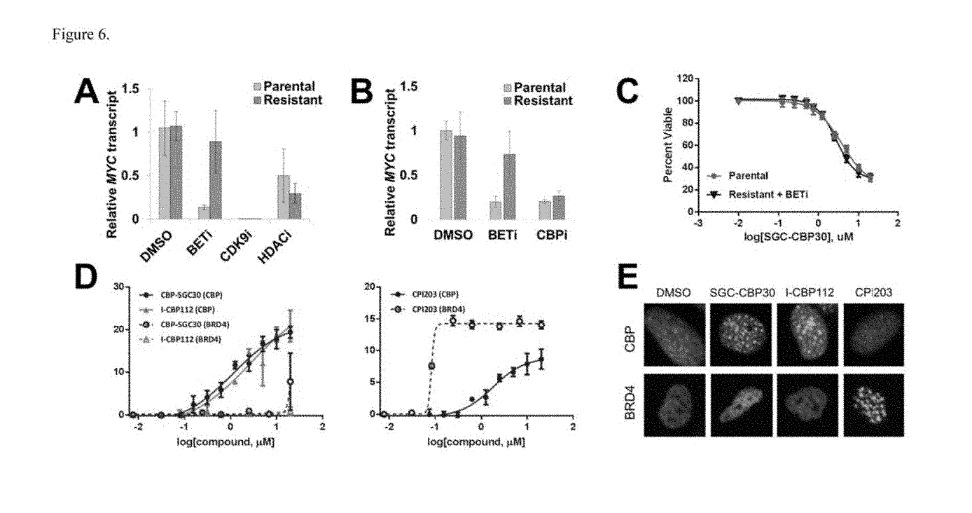

A. Parental or resistant (+0.18 μM CPI203) cells were treated with 2 μM CPI203 (BETi), 1 μM flavopiridol (CDK9i), or 2 μM SAHA (HDACi) for 24 hours and MYC mRNA was measured by qRT-PCR. B. As in (A); CBPi, 20 μM SGC-CBP30. C. Cells were treated with increasing concentrations of SGC-CBP30 and viability was measured after 4 days by resazurin staining. D. Cellular potency and selectivity of SGC-CBP30 and I-CBP112. Release of chromatin-bound ZsGreen-bromodomain fusion proteins in the presence of compound was monitored with high content imaging. Each curve represents the indicated compound and the indicated fusion protein (BRD4 or CBP). The number of nuclear foci increases with compound target engagement (values are mean of four fields per well of two technical replicates, +SEM). Calculated EC50 values are 1.1 μM (SGC-CBP30/CBP), 21.5 μM (SGC-CBP30/BRD4), 2.6 μM (I-CBP112/CBP), >20 μM (I-CBP112/BRD4), 0.08 μM (CPI203/BRD4), 1.8 μM (CPI203/CBP). E. Representative nuclei showing nuclear foci in the indicated assays (SGC-CBP30, 5 μM; I-CBP112, 5 μM; CPI203, 0.31 μM). Results are shown in FIG. 6.

### Example 6

### CBP/EP300 Bromodomain Inhibitors Suppress MYC and Inhibit Growth

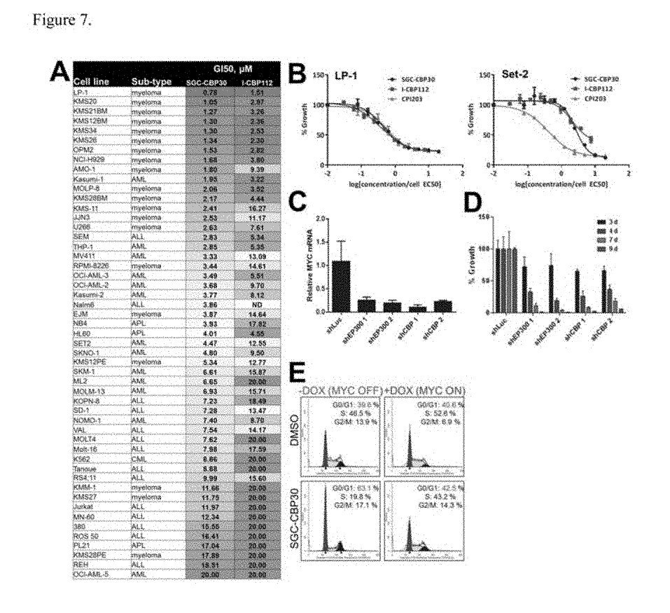

A. Growth inhibitory effects of SGC-CBP30 and I-CBP112 in the indicated cell lines. Cells were incubated with a titration of the compounds for 6d, and viability was measured with resazurin. B. Compound concentrations were divided by the EC50 values determined in (A) to control for variation in compound potency. Viability was measured with resazurin following a 6d (CBPi) or 4d (BETi) incubation. C-D. LP-1 cells were transduced with the indicated shRNA lentivirus, and relative MYC mRNA normalized to RPLPO was measured after 3d. Aliquots of cells were fixed, and viable cell number was assessed at 3d, 4d, 7d, and 9d post-infection. E. Ectopic MYC expression abrogates GO/G1 arrest induced by CBP/EP300 bromodomain inhibition. LP-1/MYC cells were incubated −/+doxycycline for 3 d. DMSO or SGC-CBP30 (2.5 μM) was added for 24 h and cells were fixed for cell cycle analysis. Results are shown in FIG. 7.

In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

All documents cited herein are incorporated by reference.

While a number of embodiments have been described, these examples may be altered to provide other embodiments that utilize the compounds and methods described herein. Therefore, the scope of this invention is to be defined by the appended claims rather than by the specific embodiments that have been represented by way of example.

>sp|Q92793|CBP_HUMAN| CREB-binding protein
OS = Homo sapiens GN = CREBBP PE = 1 SV = 3
SEQ ID NO: 1
MAENLLDGPPNPKRAKLSSPGFSANDSTDFGSLFDLENDLPDELIPNGG
ELGLLNSGNLVPDAASKHKQLSELLRGGSGSSINPGIGNVSASSPVQQG
LGGQAQGQPNSANMASLSAMGKSPLSQGDSSAPSLPKQAASTSGPTPAA
SQALNPQAQKQVGLATSSPATSQTGPGICMNANFNQTHPGLLNSNSGHS
LINQASQGQAQVMNGSLGAAGRGRGAGMPYPTPAMQGASSSVLAETLTQ
VSPQMTGHAGLNTAQAGGMAKMGITGNTSPFGQPFSQAGGQPMGATGVN
PQLASKQSMVNSLPTFPTDIKNTSVTNVPNMSQMQTSVGIVPTQAIATG
PTADPEKRKLIQQQLVLLLHAHKCQRREQANGEVRACSLPHCRTMKNVL
NHMTHCQAGKACQVAHCASSRQIISHWKNCTRHDCPVCLPLKNASDKRN
QQTILGSPASGIQNTIGSVGTGQQNATSLSNPNPIDPSSMQRAYAALGL
PYMNQPQTQLQPQVPGQQPAQPQTHQQMRTLNPLGNNPMNIPAGGITTD
QQPPNLISESALPTSLGATNPLMNDGSNSGNIGTLSTIPTAAPPSSTGV
RKGWHEHVTQDLRSHLVHKLVQAIFPTPDPAALKDRRMENLVAYAKKVE
GDMYESANSRDEyyHLLAEKIYKIQKELEEKRRSRLHKQGILGNQPALP
APGAQPPVIPQAQPVRPPNGPLSLPVNRMQVSQGMNSFNPMSLGNVQLP
QAPMGPRAASPMNHSVQMNSMGSVPGMAISPSRMPQPPNMMGAHTNNMM
AQAPAQSQFLPQNQFPSSSGAMSVGMGQPPAQTGVSQGQVPGAALPNPL
NMLGPQASQLPCPPVTQSPLHPTPPPASTAAGMPSLQHTTPPGMTPPQP
AAPTQPSTPVSSSGQTPTPTPGSVPSATQTQSTPTVQAAAQAQVTPQPQ
TPVQPPSVATPQSSQQQPTPVHAQPPGTPLSQAAASIDNRVPTPSSVAS
AETNSQQPGPDVPVLEMKTETQAEDTEPDPGESKGEPRSEMMEEDLQGA
SQVKEETDIAEQKSEPMEVDEKKPEVKVEVKEEEESSSNGTASQSTSPS
QPRKKIFKPEELRQALMPTLEALYRQDPESLPFRQPVDPQLLGIPDYFD
IVKNPMDLSTIKRKLDTGQYQEPWQYVDDVWLMFNNAWLYNRKTSRVYK
FCSKLAEVFEQEIDPVMQSLGYCCGRKYEFSPQTLCCYGKQLCTIPRDA
AYYSYQNRYHFCEKCFTEIQGENVTLGDDPSQPQTTISKDQFEKKKNDT
LDPEPFVDCKECGRKMHQICVLHYDIIWPSGFVCDNCLKKTGRPRKENK
FSAKRLQTTRLGNHLEDRVNKFLRRQNHPEAGEVFVRVVASSDKTVEVK
PGMKSRFVDSGEMSESFPYRTKALFAFEEIDGVDVCFFGMHVQEYGSDC
PPPNTRRVYISYLDSIHFFRPRCLRTAVYHEILIGYLEYVKKLGYVTGH
IWACPPSEGDDYIFHCHPPDQKIPKPKRLQEWYKKMLDKAFAERIIHDY
KDIFKQATEDRLTSAKELPYFEGDFWPNVLEESIKELEQEEEERKKEES
TAASETTEGSQGDSKNAKKKNNKKTNKNKSSISRANKKKPSMPNVSNDL
SQKLYATMEKHKEVFFVIHLHAGPVINTLPPIVDPDPLLSCDLMDGRDA
FLTLARDKHWEFSSLRRSKWSTLCMLVELHTQGQDRFVYTCNECKHHVE
TRWHCTVCEDYDLCINCYNTKSHAHKMVKWGLGLDDEGSSQGEPQSKSP
QESRRLSIQRCIQSLVHACQCRNANCSLPSCQKMKRVVQHTKGCKRKTN
GGCPVCKQLIALCCYHAKHCQENKCPVPFCLNIKHKLRQQQIQHRLQQA
QLMRRRMATMNTRNVPQQSLPSPTSAPPGTPTQQPSTPQTPQPPAQPQP
SPVSMSPAGFPSVARTQPPTTVSTGKPTSQVPAPPPPAQPPPAAVEAAR
QIEREAQQQQHLYRVNINNSMPPGRTGMGTPGSQMAPVSLNVPRPNQVS
GPVMPSMPPGQWQQAPLPQQQPMPGLPRPVISMQAQAAVAGPRMPSVQP
PRSISPSALQDLLRTLKSPSSPQQQQQVLNILKSNPQLMAAFIKQRTAK
YVANQPGMQPQPGLQSQPGMQPQPGMHQQPSLQNLNAMQAGVPRPGVPP
QQQAMGGLNPQGQALNIMNPGHNPNMASMNPQYREMLRRQLLQQQQQQQ
QQQQQQQQQQQGSAGMAGGMAGHGQFQQPQGPGGYPPAMQQQQRMQQHL
PLQGSSMGQMAAQMGQLGQMGQPGLGADSTPNIQQALQQRILQQQQMKQ
QIGSPGQPNPMSPQQHMLSGQPQASHLPGQQIATSLSNQVRSPAPVQSP
RPQSQPPHSSPSPRIQPQPSPHHVSPQTGSPHPGLAVTMASSIDQGHLG
NPEQSAMLPQLNTPSRSALSSELSLVGDTTGDTLEKFVEGL
>sp|Q09472|EP300_HUMAN Histone acetyltransferase
p300 OS = Homo sapiens GN = EP300 PE = 1 SV = 2
SEQ ID NO: 2
MAENVVEPGPPSAKRPKLSSPALSASASDGTDFGSLFDLEHDLPDELIN
STELGLTNGGDINQLQTSLGMVQDAASKHKQLSELLRSGSSPNLNMGVG
GPGQVMASQAQQSSPGLGLINSMVKSPMTQAGLTSPNMGMGTSGPNQGP
TQSTGMMNSPVNQPAMGMNTGMNAGMNPGMLAAGNGQGIMPNQVMNGSI
GAGRGRQNMQYPNPGMGSAGNLLTEPLQQGSPQMGGQTGLRGPQPLKMG
MMNNPNPYGSPYTQNPGQQIGASGLGLQIQTKTVLSNNLSPFAMDKKAV
PGGGMPNMGQQPAPQVQQPGLVTPVAQGMGSGAHTADPEKRKLIQQQLV
LLLHAHKCQRREQANGEVRQCNLPHCRTMKNVLNHMTHCQSGKSCQVAH
CASSRQIISHWKNCTRHDCPVCLPLKNAGDKRNQQPILTGAPVGLGNPS
SLGVGQQSAPNLSTVSQIDPSSIERAYAALGLPYQVNQMPTQPQVQAKN
QQNQQPGQSPQGMRPMSNMSASPMGVNGGVGVQTPSLLSDSMLHSAINS
QNPMMSENASVPSLGPMPTAAQPSTTGIRKQWHEDITQDLRNHLVHKLV
QAIFPTPDPAALKDRRMENLVAYARKVEGDMYESANNRAEYYHLLAEKI
YKIQKELEEKRRTRLQKQNMLPNAAGMVPVSMNPGPNMGQPQPGMTSNG
PLPDPSMIRGSVPNQMMPRITPQSGLNQFGQMSMAQPPIVPRQTPPLQH
HGQLAQPGALNPPMGYGPRMQQPSNQGQFLPQTQFPSQGMNVTNIPLAP
SSGQAPVSQAQMSSSSCPVNSPIMPPGSQGSHIHCPQLPQPALHQNSPS
PVPSRTPTPHHTPPSIGAQQPPATTIPAPVPTPPAMPPGPQSQALHPPP
RQTPTPPTTQLPQQVQPSLPAAPSADQPQQQPRSQQSTAASVPTPTAPL
LPPQPATPLSQPAVSIEGQVSNPPSTSSTEVNSQAIAEKQPSQEVKMEA
KMEVDQPEPADTQPEDISESKVEDCKMESTETEERSTELKTEIKEEEDQ
PSTSATQSSPAPGQSKKKIFKPEELRQALMPTLEALYRQDPESLPFRQP
VDPQLLGIPDYFDIVKSPMDLSTIKRKLDTGQYQEPWQYVDDIWLMFNN
AWLYNRKTSRVYKYCSKLSEVFEQEIDPVMQSLGYCCGRKLEFSPQTLC
CYGKQLCTIPRDATYYSYQNRYHFCEKCFNEIQGESVSLGDDPSQPQTT
INKEQFSKRKNDTLDPELFVECTECGRKMHQICVLHHEIIWPAGFVCDG
CLKKSARTRKENKFSAKRLPSTRLGTFLENRVNDFLRRQNHPESGEVTV
RVVHASDKTVEVKPGMKARFVDSGEMAESFPYRTKALFAFEEIDGVDLC
FFGMHVQEYGSDCPPPNQRRVYISYLDSVHFFRPKCLRTAVYHEILIGY
LEYVKKLGYTTGHIWACPPSEGDDYIFHCHPPDQKIPKPKRLQEWYKKM
LDKAVSERIVHDYKDIFKQATEDRLTSAKELPYFEGDFWPNVLEESIKE
LEQEEEERKREENTSNESTDVTKGDSKNAKKKNNKKTSKNKSSLSRGNK
KKPGMPNVSNDLSQKLYATMEKHKEVFFVIRLIAGPAANSLPPIVDPDP
LIPCDLMDGRDAFLTLARDKHLEFSSLRRAQWSTMCMLVELHTQSQDRF
VYTCNECKHHVETRWHCTVCEDYDLCITCYNTKNHDHKMEKLGLGLDDE
SNNQQAAATQSPGDSRRLSIQRCIQSLVHACQCRNANCSLPSCQKMKRV
VQHTKGCKRKTNGGCPICKQLIALCCYHAKHCQENKCPVPFCLNIKQKL
RQQQLQHRLQQAQMLRRRMASMQRTGVVGQQQGLPSPTPATPTTPTGQQ
PTTPQTPQPTSQPQPTPPNSMPPYLPRTQAAGPVSQGKAAGQVTPPTPP
QTAQPPLPGPPPAAVEMAMQIQRAAETQRQMAHVQIFQRPIQHQMPPMT
PMAPMGMNPPPMTRGPSGHLEPGMGPTGMQQQPPWSQGGLPQPQQLQSG
MPRPAMMSVAQHGQPLNMAPQPGLGQVGISPLKPGTVSQQALQNLLRTL
RSPSSPLQQQQVLSILHANPQLLAAFIKQRAAKYANSNPQPIPGQPGMP
QGQPGLQPPTMPGQQGVHSNPAMQNMNPMQAGVQRAGLPQQQPQQQLQP
PMGGMSPQAQQMNMNHNTMPSQFRDILRRQQMMQQQQQQGAGPGIGPGM
ANHNQFQQPQGVGYPPQQQQRMQHHMQQMQQGNMGQIGQLPQALGAEAG
ASLQAYQQRLLQQQMGSPVQPNPMSPQQHMLPNQAQSPHLQGQQIPNSL
SNQVRSPQPVPSPRPQSQPPHSSPSPRMQPQPSPHHVSPQTSSPHPGLV
AAQANPMEQGHFASPDQNSMLSQLASNPGMANLHGASATDLGLSTDNSD
LNSNLSQSTLDIH

1. A method for treating or delaying progression of cancer in an individual comprising administering an effective amount of a CBP/EP300 inhibitor and a BET inhibitor to the individual.

2. The method of claim 1, wherein the CBP/EP300 inhibitor and the BET inhibitor are concomitantly administered.

3. The method of claim 1, wherein the CBP/EP300 inhibitor and the BET inhibitor are co-formulated.

4. The method of claim 1, wherein the CBP/EP300 inhibitor is administered separately from the BET inhibitor.

5. The method of claim 1, wherein the CBP/EP300 inhibitor is administered sequentially with the BET inhibitor.

6. The method of claim 1, wherein the CBP/EP300 inhibitor is administered simultaneously with the BET inhibitor.

7. The method of claim 1, wherein the individual is administered the BET inhibitor and subsequently administered the CBP/EP300 inhibitor.

8. The method of claim 1, wherein the individual is administered the CBP/EP300 inhibitor and subsequently administered the BET inhibitor.

9. The method of claim 1 any one of claims 1 8, wherein administration of the CBP/EP300 inhibitor and the BET inhibitor slows growth of cancer cells to a greater extent than administration of either inhibitor alone.

10. A method of treating or delaying progression of cancer, wherein the cancer is resistant to a BET inhibitor, in an individual comprising administering an effective amount of a CBP/EP300 inhibitor to the individual.

11. The method of claim 1, wherein the cancer is selected from acoustic neuroma, acute leukemia, acute lymphocytic leukemia, acute myelocytic leukemia, acute t-cell leukemia, B-cell proliferative cancer, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer (NSCLC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

12. The method of claim 1, wherein the cancer is a B-cell proliferative cancer.

13. The method of claim 12, wherein the cancer is leukemia or lymphoma.

14. The method of claim 12, wherein the cancer is leukemia.

15. The method of claim 1, wherein the cancer is breast cancer.

16. The method of claim 1, wherein the cancer is myeloma.

17. The method of claim 1, wherein the individual is human.

18. The method of claim 1, wherein the CBP/EP300 inhibitor is a HAT domain inhibitor.

19. The method claim 1, wherein the CBP/EP300 inhibitor is a bromodomain inhibitor.

20. The method of claim 1, wherein the CBP/EP300 inhibitor inhibits CBP.

21. The method of claim 1, wherein the EP300 inhibitor inhibits EP300.

22. A CBP/EP300 inhibitor and BET inhibitor combination for use in medical treatment or diagnosis including therapy and/or treating cancer.

23. A CBP/EP300 inhibitor for use in medical treatment or diagnosis including therapy and/or treating cancer, wherein the cancer is resistant to a BET inhibitor.
